(19) United States
(12) Patent Application Publication
(10) Pub. No.: US 2026/0224297 A1
(43) Pub. Date: Aug. 6, 2026

(54) ULTRASOUND IMAGING SYSTEM AND METHOD FOR GUIDING INTERVENTIONAL DEVICES, AND MEDICAL SYSTEM

(71) Applicant: GE Precision Healthcare LLC, Waukesha, WI (US)

(72) Inventors: Ke Tao, Wuxi (CN); Shangjie Du, Wuxi (CN)

(21) Appl. No.: 19/458,802

(22) Filed: Jan. 24, 2026

(30) Foreign Application Priority Data
Jan. 24, 2025 (CN) 202510122423.9

(51) Int. Cl. A61B 34/20; A61B 8/00; A61B 8/08

(52) U.S. Cl. CPC A61B 34/20; A61B 8/0841; A61B 8/4461; A61B 8/54; A61B 2034/2063

(57) ABSTRACT

An ultrasound imaging assembly including a housing, an ultrasonic transducer movable within the housing, and a controller configured to perform the following: moving the ultrasonic transducer within the housing and acquiring an image sequence of an object being scanned, the image sequence including a plurality of parallel first ultrasound images; determining position information of a region of interest according to the image sequence; moving, according to the position information, the ultrasonic transducer to a first position corresponding to the region of interest and acquiring a second ultrasound image in real time; and generating, according to the second ultrasound image, indication information for indicating a positional relationship between an end position of the interventional object and the region of interest.

### CROSS-REFERENCE TO RELATED APPLICATION

This application claim priority to Chinese Patent Application No. 202510122423.9, which was file on January 24, 2025 at the Chinese Patent Office. The entire contents of the above-listed application are incorporated by reference herein in their entirety.

### TECHNICAL FIELD

Embodiments of the present application relate to the technical field of ultrasound imaging, and relate in particular to an ultrasound imaging system for guiding an interventional object, a method, and a medical system.

### BACKGROUND

Ultrasound imaging technology is a real-time, non-destructive imaging technique that uses an ultrasound imaging assembly to send an ultrasonic signal to an object being scanned and receive an ultrasonic echo signal from the object, and processes the ultrasonic echo signal to perform imaging. In some application scenarios, ultrasound imaging can provide indication information related to the position of an interventional object during an interventional procedure (for example, biopsy or ablation).

### SUMMARY

The inventors have found that for a conventional handheld probe-type ultrasound imaging system, because handheld operation is easily affected by factors such as the operator's hand tremors and insufficient operation experience, it is difficult to ensure that the probe can accurately move to a position corresponding to the region of interest. During an interventional procedure, the pressure and angle of the probe in contact with the surface of the object being

scanned may be unstable due to changes in the handheld manner, which affects the quality and consistency of ultrasound images.

For example, when the interventional object approaches the region of interest, accurate images are required to determine the positional relationship, but the handheld probe may change propagation and reflection characteristics of ultrasonic signals due to changes in the pressure or angle, causing structures displayed in the images to be unclear or distorted. As a result, the positional relationship between the end position of the interventional object and the region of interest cannot be reliably determined.

In view of at least one of the above problems, embodiments of the present application provide an ultrasound imaging system for guiding an interventional object, a method, and a medical system.

According to an aspect of the embodiments of the present application, an ultrasound imaging system for guiding an interventional object is provided, the system comprising an ultrasound imaging assembly and a controller, and the ultrasound imaging assembly comprising a housing and an ultrasonic transducer movable within the housing; the controller is configured to perform the following: moving the ultrasonic transducer within the housing and acquiring an image sequence of an object being scanned, the image sequence comprising a plurality of parallel first ultrasound images; determining position information of a region of interest according to the image sequence; moving, according to the position information, the ultrasonic transducer to a first position corresponding to the region of interest and acquiring a second ultrasound image in real time; and generating, according to the second ultrasound image, indication information for indicating a positional relationship between an end position of the interventional object and the region of interest.

According to another aspect of the embodiments of the present application, a medical system is provided. The medical system comprises: the ultrasound imaging system described above; and an interventional assembly comprising the interventional object.

According to yet another aspect of the embodiments of the present application, an imaging method for an ultrasound imaging system is provided, the ultrasound imaging system comprising an ultrasound imaging assembly, and the ultrasound imaging assembly comprising a housing and an ultrasonic transducer movable within the housing. The method comprises: moving the ultrasonic transducer within the housing and acquiring an image sequence of an object being scanned, the image sequence comprising a plurality of parallel first ultrasound images; determining position information of a region of interest according to the image sequence; moving, according to the position information, the ultrasonic transducer to a first position corresponding to the region of interest and acquiring a second ultrasound image in real time; and generating, according to the second ultrasound image, indication information for indicating a positional relationship between an end position of an interventional assembly and the region of interest.

One of the beneficial effects of the embodiments of the present application is that: an image sequence of an object being scanned is acquired by moving the ultrasonic transducer within the housing; position information of a region of interest is determined according to the image sequence; the ultrasonic transducer is automatically moved to a first position corresponding to the region of interest according to the position information and a second ultrasound image is acquired in real time at the first position; and indication information for indicating a positional relationship between an end position of the interventional object and the region of interest is generated according to the second ultrasound image. Hence, the ultrasonic transducer can be accurately moved to the first position corresponding to the region of interest, and when the interventional object reaches a scanning plane corresponding to the first position, the positional relationship between the end position of the interventional object and the region of interest can be reliably determined according to the second ultrasound image acquired in real time at the first position, thereby helping the operator to accurately understand the result of the interventional procedure.

With reference to the following description and drawings, specific implementations of the embodiments of the present application are disclosed in detail, and the way in which the principles of the embodiments of the present application can be employed are illustrated. It should be understood that the implementations of the present application are not limited in scope thereby. Within the scope of the spirit and clauses of the appended claims, the implementations of the present application comprise many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and those of ordinary skill in the art may obtain other implementations according to the drawings without involving inventive effort. In the drawings:

FIG. 1 is a schematic diagram of the structure of an ultrasound imaging system according to an embodiment of the present application;

FIG. 2 is a schematic diagram of an ultrasound imaging assembly according to an embodiment of the present application;

FIG. 3 is a schematic diagram of internal signal interaction in an ultrasound imaging system according to an embodiment of the present application;

FIG. 4 is a schematic diagram of a control method executed by a controller according to an embodiment of the present application;

FIG. 5 is a schematic diagram of an image sequence according to an embodiment of the present application;

FIG. 6 is a schematic diagram of a method for determining position information of a region of interest according to an embodiment of the present application;

FIG. 7 is a schematic diagram of a third ultrasound image according to an embodiment of the present application;

FIG. 8 is a schematic diagram of a coronal image and a region of interest according to an embodiment of the present application;

FIG. 9 is a schematic diagram of an ultrasound slice sequence and a region of interest according to an embodiment of the present application;

FIG. 10 is a schematic diagram of a method for determining a first position according to an embodiment of the present application;

FIG. 11 is a schematic diagram of an ultrasound slice and a region of interest according to an embodiment of the present application;

FIG. 12 is a schematic diagram of a second ultrasound image according to an embodiment of the present application;

FIG. 13 is a schematic diagram of guidance information according to an embodiment of the present application;

FIG. 14 is a schematic diagram of the structures of an ultrasonic transducer and an interventional assembly according to an embodiment of the present application;

FIG. 15 is a schematic diagram of a positional relationship between an interventional assembly, an ultrasonic transducer, and a region of interest according to an embodiment of the present application; and

FIG. 16 is a schematic diagram of an interventional assembly, a housing, an ultrasonic transducer, and a second ultrasound image according to an embodiment of the present application.

### DETAILED DESCRIPTION

The aforementioned and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first”, “second”, etc., are used to distinguish different elements, but do not represent a spatial arrangement or temporal order, etc., of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies.

In the embodiments of the present application, the singular forms “a”, “the”, etc., include plural forms, and should be broadly construed as “a type of” or “a class of” rather than being limited to the meaning of “one”. In addition, the term “the” should be construed as including both the singular and plural forms, unless otherwise specified in the context. In addition, the term “according to” should be construed as “at least partially according to ...” and the term “based on” should be construed as “at least partially based on ...”, unless otherwise explicitly specified in the context.

The features described and/or illustrated for one implementation may be used in one or more other implementations in the same or similar way, be combined with features in other implementations, or replace features in other implementations. The terms “include/comprise” when used herein refer to the presence of features, integrated components, steps, or assemblies, but do not preclude the presence or addition of one or more other features, integrated components, steps, or assemblies.

In some embodiments, an ultrasound imaging system may be used to perform scanning and imaging on a body part of a human body or other living entities. However, the present application is not limited thereto, and the ultrasound imaging system may also be used to perform scanning and imaging on a non-living entity. Moreover, the present application is also applicable to other imaging apparatuses having similar structures or functions (for example, apparatuses for performing imaging by applying visible light, X-rays, a magnetic field, or other physical signals to an object being scanned).

The following is a specific description of the embodiments of the present application with reference to the drawings.

Embodiments of the present application provide an ultrasound imaging system for guiding an interventional object.

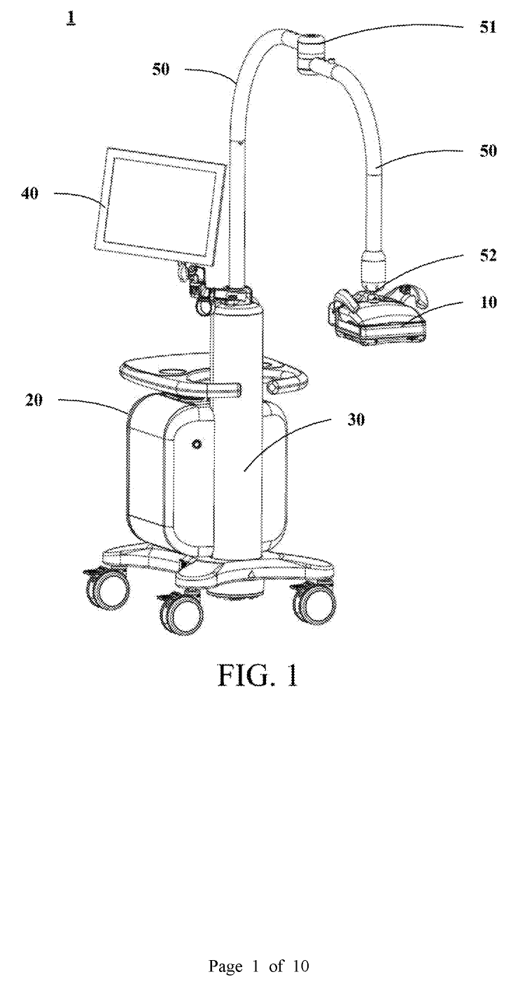

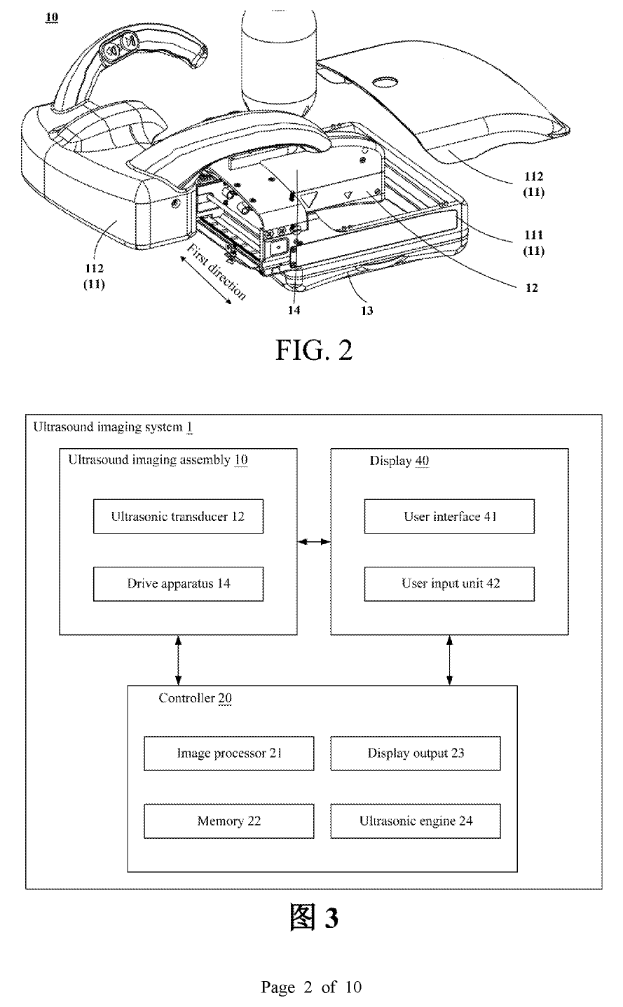

FIG. 1 is a schematic diagram of the structure of an ultrasound imaging system according to an embodiment of the present application. As shown in FIG. 1, the ultrasound scanning system 1 includes an ultrasound imaging assembly 10 and a controller 20. FIG. 2 is a schematic diagram of an ultrasound imaging assembly according to an embodiment of the present application. As shown in FIG. 2, the ultrasound imaging assembly 10 includes a housing 11 and an ultrasonic transducer 12.

The controller 20 is configured to perform the following: moving the ultrasonic transducer 12 within the housing 11 and acquiring an image sequence of an object being scanned, the image sequence including a plurality of parallel first ultrasound images; determining position information of a region of interest according to the image sequence; moving, according to the position information, the ultrasonic transducer 12 to a first position corresponding to the region of interest and acquiring a second ultrasound image in real time; and generating, according to the second ultrasound image, indication information for indicating a positional relationship between an end position of an interventional object and the region of interest.

According to the above embodiment, an image sequence of an object being scanned is acquired by moving the ultrasonic transducer 12 within the housing 11; position information of a region of interest is determined according to the image sequence; the ultrasonic transducer 12 is automatically moved to a first position corresponding to the region of interest according to the position information and a second ultrasound image is acquired in real time at the first position; and indication information for indicating a positional relationship between an end position of the interventional object and the region of interest is generated according to the second ultrasound image. Hence, the ultrasonic transducer 12 can be accurately moved to the first position corresponding to the region of interest, and when the interventional object reaches a scanning plane corresponding to the first position, the positional relationship between the end position of the interventional object and the region of interest can be reliably determined according to the second ultrasound image, thereby helping the operator to accurately understand the result of the interventional procedure.

In some embodiments, the ultrasound imaging system 1 may be in various forms. For example, the ultrasound imaging system 1 may be a full-field breast ultrasound imaging system. The structure of the ultrasound imaging system 1 will be exemplarily described below with reference to the accompanying drawings by taking a full-field breast ultrasound imaging system as an example.

As shown in FIG. 1, in addition to the ultrasound imaging assembly 10 and the controller 20, the ultrasound imaging system 1 may further include a main body frame 30 and a movable support arm 50 including a hinge joint 51 or other similar structures. One end of the support arm 50 is connected to the main body frame 30, and the other end is connected to the ultrasound imaging assembly 10 through a ball-and-socket connector 52 or other similar structures.

In some embodiments, the support arm 50 may be configured to enable the ultrasound imaging assembly 10 to apply a preset pressure to the object being scanned (for example, a breast) during scanning, thereby ensuring that the ultrasound imaging assembly 10 is in close contact with the object being scanned. In this way, ultrasonic signals can be sent and/or received with low attenuation, and relative displacement between the ultrasound imaging assembly 10 and the object being scanned can be avoided, thereby helping to improve imaging quality.

As shown in FIG. 1, the ultrasound imaging system 1 may further include a display 40. The display 40 may be connected to the main body frame 30, and thus the weight of the support arm 50 and the balance mechanism of the support arm 50 would not be affected. Although FIG. 1 shows the display 40 being connected to the main body frame 30, in other examples, the display 40 may be connected to other components of the ultrasound imaging system 1.

As shown in FIG. 2, the ultrasound imaging assembly 10 may further include a film assembly 13. The film assembly 13 includes an outer frame and a film. The film is fixedly disposed in the outer frame, and the outer frame is detachably connected to other components (for example, a framework 111 of the housing 11 to be described later) of the ultrasound imaging assembly 10.

During scanning and imaging of the ultrasound imaging system, one surface of the film is at least partially in contact with the ultrasonic transducer 12 of the ultrasound imaging assembly 10, and the other surface of the film is at least partially in contact with the object being scanned. In this way, the ultrasonic transducer 12 can be ensured to send and receive signals with low attenuation, and the object being scanned can be fixed to facilitate scanning. In some embodiments, the film may be a tensioned fabric sheet.

As shown in FIG. 2, the housing 11 includes a framework 111, and the ultrasonic transducer 12 moves within a space enclosed by the framework 111 to cover a preset scanning range. The ultrasonic transducer 12 may move within the framework 111 in various manners. For example, as shown in FIG. 2, the ultrasonic transducer 12 reciprocates in a first direction. Alternatively, the ultrasonic transducer 12 rotates about a preset center of circle, or performs a movement combining various motion types, and so on.

As shown in FIG. 2, the housing 11 may further include an upper housing 112 disposed on the upper side of the framework 111. The upper side of the upper housing 112 faces the support arm 50.

As shown in FIG. 2, the ultrasound imaging assembly 10 may further include a drive apparatus 14 for driving the ultrasonic transducer 12 to move. The drive apparatus 14 may be disposed on top of the ultrasonic transducer 12, but the present application is not limited thereto. The drive apparatus 14 may alternatively be disposed at other positions of the ultrasonic transducer 12 or connected to the ultrasonic transducer 12 in other manners.

FIG. 3 is a schematic diagram of internal signal interaction in an ultrasound imaging system according to an embodiment of the present application. As shown in FIG. 3, in the ultrasound imaging system 1, there is signal interaction between the ultrasound imaging assembly 10, the controller 20, and the display 40. The ultrasound imaging assembly 10, the controller 20, and the display 40 may be independent components that communicate with each other. However, the present application is not limited thereto, and one or more of these components may also be integrated.

In the ultrasound imaging assembly 10, the ultrasonic transducer 12 may include a transducer array including a plurality of transducer elements. The transducer elements can convert electrical energy into ultrasonic waves for transmission and can detect reflected ultrasonic echoes. The ultrasound imaging assembly 10 can communicate with the controller 20 to send raw scan data (for example, ultrasonic echo signals) to the controller 20.

The controller 20 may include, for example, an image processor 21, a memory 22, display output 23, and an ultrasonic engine 24. The memory 22 can store various types of information. In addition, the memory 22 can further store a control program, and the program can be executed under the control of the image processor 21 or the ultrasonic engine 24.

The image processor 21 can process the received raw scan data to form a displayable image of a tissue sample, for example, to form an image sequence including a plurality of ultrasound slices. But the present application is not limited thereto. The raw scan data may alternatively be processed by a remote processor or the like.

The ultrasonic engine 24 can activate the transducer elements of the ultrasonic transducer 12. In some embodiments, the ultrasonic engine 24 may further activate the drive apparatus 14 in the ultrasound imaging assembly. In addition, the ultrasonic engine 24 may also integrate related functions of the image processor 21.

The controller 20 can communicate with the display 40 via the display output 23, and transmit image data of the displayable image from the ultrasonic engine 24 or the image processor 21 to the display 40. The display 40 may include a user interface 41 configured to display images or other information to an operator. The display 40 may further include a user input unit 42 for receiving operator input. In one example, the user input unit 42 may be a touchscreen of the display 40. However, other types of user input mechanisms are also possible, such as a mouse or a keyboard.

The structure and internal signal interaction of the ultrasound imaging system 1 are exemplarily described above by taking a full-field breast ultrasound imaging system as an example. However, the present application is not limited thereto, and the ultrasound imaging system 1 may also be other types of ultrasound imaging systems.

The actions of the controller 20 in this embodiment of the present application will be exemplarily described below.

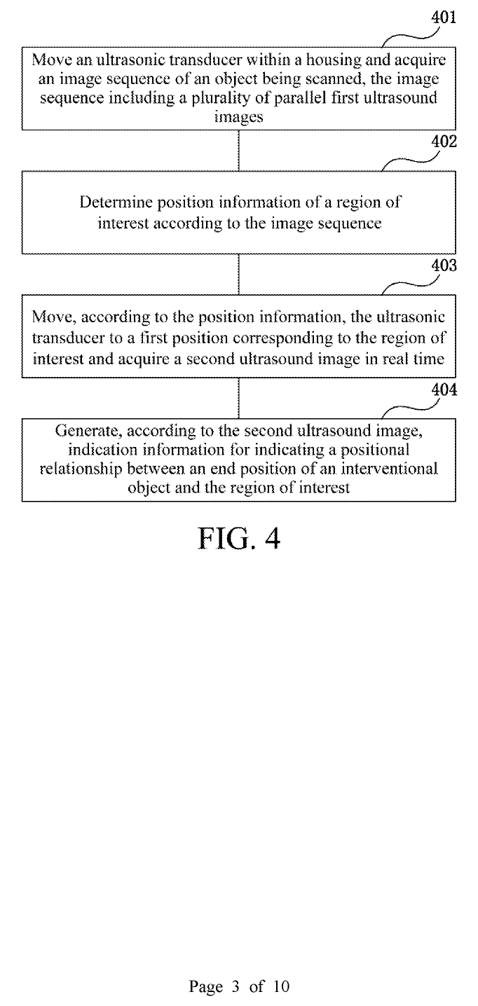

FIG. 4 is a schematic diagram of a control method executed by a controller according to an embodiment of the present application. As shown in FIG. 4, the control method includes:

Step 401: moving the ultrasonic transducer within the housing and acquiring an image sequence of an object being scanned, the image sequence including a plurality of parallel first ultrasound images;

Step 402: determining position information of a region of interest according to the image sequence;

Step 403: moving, according to the position information, the ultrasonic transducer to a first position corresponding to the region of interest and acquiring a second ultrasound image in real time; and and

Step 404: generating, according to the second ultrasound image, indication information for indicating a positional relationship between an end position of the interventional object and the region of interest.

In step 401, while moving within the housing, the ultrasonic transducer can acquire first ultrasound images at different positions in the movement direction in accordance with a preset frame interval. In other words, the different first ultrasound images in the image sequence may correspond to different positions of the ultrasonic transducer in the movement direction.

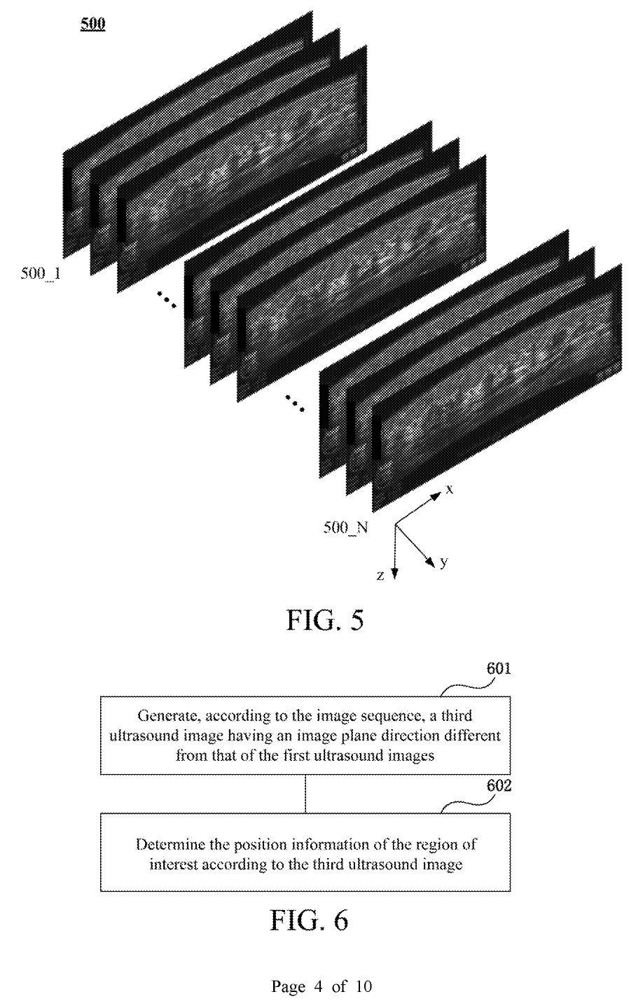

FIG. 5 is a schematic diagram of an image sequence according to an embodiment of the present application. As shown in FIG. 5, an image sequence 500 includes N parallel first ultrasound images 500_1 to 500_N, where N is an integer greater than or equal to 1. For example, the ultrasonic transducer reciprocates within the housing in the y-axis direction shown in FIG. 5, and the range of motion is from position 1 to position 2, wherein the first ultrasound image 500_1 may correspond to position 1 of the ultrasonic transducer, the first ultrasound image 500_N may correspond to position 2 of the ultrasonic transducer, and the first ultrasound images between the first ultrasound images 500_1 and 500_Nrespectively correspond to various positions between position 1 and position 2. But the present application is not limited thereto, and the first ultrasound images and the positions of the ultrasonic transducer may alternatively have other corresponding relationships.

In step 402, position information of a region of interest is determined according to the image sequence.

The position information of the region of interest may be in various forms. For example, the position information of the region of interest may be information of first ultrasound images in the image sequence that include the region of interest. For example, the position information may be represented by the serial numbers of the first ultrasound images in the image sequence that include the region of interest. But the present application is not limited thereto, and the position information of the region of interest may further include other information, such as position information of the region of interest within a first ultrasound image.

In some embodiments, the position information of the region of interest may be determined in various manners. For example, the position information of the region of interest may be determined directly according to the first ultrasound images in the image sequence. Specifically, detection and recognition can be performed on the respective first ultrasound images in the image sequence to determine a set of first ultrasound images including the region of interest, and the serial numbers of these first ultrasound images are used as the position information of the region of interest.

In some other embodiments, the first ultrasound images in the image sequence can be processed, and the position information of the region of interest can be determined according to the processed images.

FIG. 6 is a schematic diagram of a method for determining position information of a region of interest according to an embodiment of the present application. As shown in FIG. 6, the method for determining position information of a region of interest includes:

Step 601: generating, according the image sequence, a third ultrasound image having an image plane direction different from that of the first ultrasound images; and

Step 602: determining the position information of the region of interest according to the third ultrasound image.

The position information of the region of interest can be accurately determined by analyzing ultrasound images with different image plane directions. In addition, during determination of the position information of the region of interest, it is not necessary to perform detection and recognition on the respective first ultrasound images in the image sequence. Therefore, the amount of data to be processed can be reduced, and computational efficiency can be improved.

In step 601, the third ultrasound image can be generated according to the image sequence by using various image reconstruction techniques. The image plane direction of the first ultrasound images and the image plane direction of the third ultrasound image may be perpendicular to each other.

For example, the first ultrasound images may be perpendicular to a plane in which the object being scanned is located, and the third ultrasound image may be parallel to the plane in which the object being scanned is located. For example, as shown in FIG. 5, the image planes of the first ultrasound images are parallel to the plane xz, and the plane in which the object being scanned is located is parallel to the plane xy, wherein the plane xz is perpendicular to the plane zy. By using a breast as an example of the object being scanned, the plane in which the object is located is parallel to the coronal plane of the human body, and the image planes of the first ultrasound images may be various planes perpendicular to the coronal plane. For example, the image planes of the first ultrasound images may be parallel to the sagittal plane or the transverse plane of the human body.

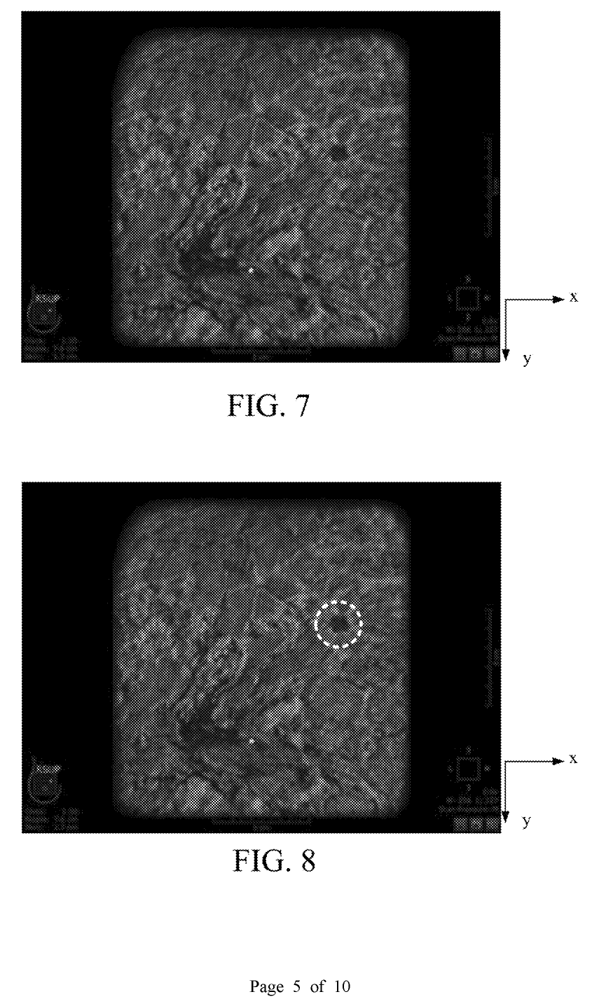

FIG. 7 is a schematic diagram of a third ultrasound image according to an embodiment of the present application. As shown in FIG. 7, an image plane of the third ultrasound image is parallel to the plane xy, and because the plane in which the object being scanned is located is also parallel to the plane xy, the third ultrasound image is parallel to the plane in which the object being scanned is located. By using a breast as an example of the object being scanned, the image plane of the third ultrasound image is parallel to the coronal plane of the human body.

In some embodiments, the third ultrasound image may be an image having a smaller data amount than the image sequence including the plurality of first ultrasound images. As an example, the third ultrasound image may be a two-dimensional image, which includes information of projections of the respective first ultrasound images on planes parallel to the plane in which the object being scanned is located. In other words, the third ultrasound image may be an image generated by compressing the respective first ultrasound images in the image sequence in the z-axis direction using an image reconstruction technique. As the first ultrasound images includes the region of interest, the region of interest is projected to the third ultrasound image, so that the third ultrasound image includes range information of the region of interest in planes parallel to the plane in which the object being scanned is located. But the present application is not limited thereto, and the third ultrasound image may also be an image in other forms.

In step 602, the position information of the region of interest may be determined according to the third ultrasound image in various manners. For example, detection and recognition are performed on the third ultrasound image to determine range information of the region of interest in the third ultrasound image. According to the range information and the correspondences between the first ultrasound images in the image sequence and the third ultrasound image, a set of first ultrasound images including the region of interest is determined.

Hereinafter, with reference to the accompanying drawings, the manner of determining the position information of the region of interest in the present application will be exemplarily described by using a breast as an example of the scanning object, transverse images of the object being scanned as an example of the first ultrasound images, and a coronal image of the object being scanned as an example of the third ultrasound image.

After a set of transverse ultrasound slices of the breast (that is, the first ultrasound images shown in FIG. 5) are acquired, this set of ultrasound slices are converted into a coronal image (that is, the third ultrasound image shown in FIG. 7) by using a post-processing algorithm (for example, an image reconstruction algorithm).

The coronal image is processed using an artificial neural network model to automatically detect and recognize the region of interest in the coronal image and to determine range information of the region of interest on the coronal image. FIG. 8 is a schematic diagram of a coronal image and a region of interest according to an embodiment of the present application. As shown in FIG. 8, the region indicated by the dashed circle is the region of interest in the coronal image.

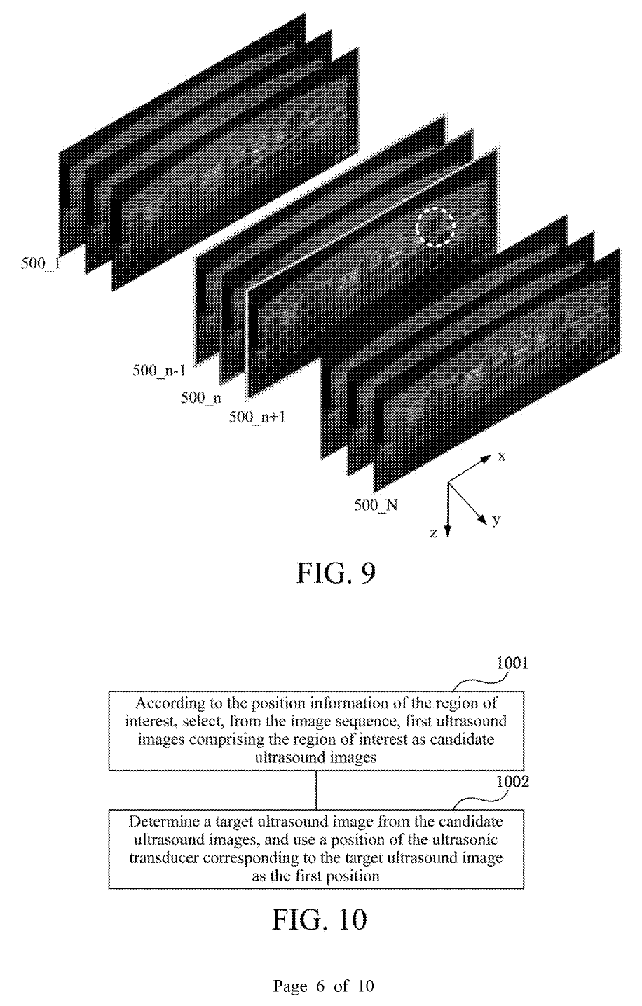

By using a post-processing algorithm (for example, an image reconstruction algorithm), the range information of the region of interest on the coronal image is inversely mapped back to the transverse ultrasound slices, thereby determining a set of ultrasound slices including the region of interest. FIG. 9 is a schematic diagram of an ultrasound slice sequence and a region of interest according to an embodiment of the present application. As shown in FIG. 9, in the sequence of ultrasound slices 500_1 to 500_N, the ultrasound slices 500_n-1, 500_n, and 500_n+1 include the region of interest (such as the region indicated by the dashed circle in the ultrasound slice 500_n+1), and the ultrasound slice serial numbers 500_n-1, 500_n, and 500_n+1 are used as the position information of the region of interest.

In step 403, a first position corresponding to the region of interest can be determined according to the position information of the region of interest. The first position may be a position of the ultrasonic transducer in the movement direction. The first position may be determined in various manners.

FIG. 10 is a schematic diagram of a method for determining a first position according to an embodiment of the present application. As shown in FIG. 10, the method includes:

Step 1001: according to the position information of the region of interest, selecting, from the image sequence, first ultrasound images comprising the region of interest as candidate ultrasound images; and and

Step 1002: determining a target ultrasound image from the candidate ultrasound images, and using a position of the ultrasonic transducer corresponding to the target ultrasound image as the first position.

By determining the target ultrasound image from the candidate ultrasound images including the region of interest, the amount of data to be processed can be reduced, and computational efficiency can be improved.

In step 1002, the target ultrasound image may be determined in various manners. In some embodiments, detection can be performed on the candidate ultrasound images for the region of interest, and the target ultrasound image can be determined according to a region-of-interest detection result. Thus, the target ultrasound image can be selected more reliably.

The target ultrasound image may be a candidate ultrasound image in which the cross-sectional area of the region of interest is greater than a preset area threshold. For example, the target ultrasound image may be a candidate ultrasound image with the maximum cross-sectional area of the region of interest.

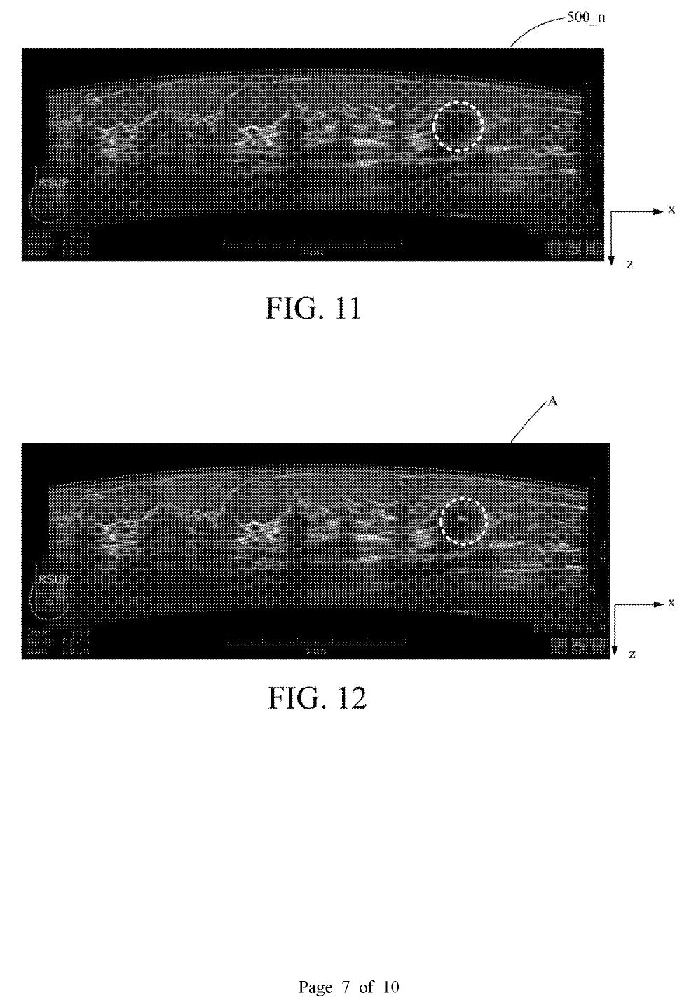

For example, as shown in FIG. 9, the ultrasound slices 500_n-1, 500_n, and 500_n+1 are used as the candidate ultrasound images including the region of interest, and the ultrasound slices 500_n-1, 500_n, and 500_n+1 separately undergo detection and recognition. FIG. 11 is a schematic diagram of an ultrasound slice and a region of interest according to an embodiment of the present application. As shown in FIG. 11, taking the ultrasound slice 500_n as an example, the region indicated by the dashed circle is the region of interest in the ultrasound slice 500_n. If the region of interest in the ultrasound slice 500_n among the ultrasound slices 500_n-1, 500_n, and 500_n+1 has the maximum cross-sectional area, then the ultrasound slice 500_n is taken as the target ultrasound slice.

For another example, the target ultrasound image may be a candidate ultrasound image in which the distance between two points on the boundary of a cross section of the region of interest is greater than a preset distance threshold. For example, the target ultrasound image may be a candidate ultrasound image in which the distance between two points on the boundary of the region of interest is the greatest. However, the present application is not limited thereto, and the target ultrasound image may alternatively be another candidate ultrasound image determined according to the region-of-interest detection result.

In some embodiments, the target ultrasound image may also be determined from the candidate ultrasound images according to ranking of the candidate ultrasound images. Thus, the target ultrasound image can be determined in a simple manner.

The target ultrasound image may be a candidate ultrasound image at the middle position among the plurality of candidate ultrasound images. But the present application is not limited thereto, and the target ultrasound image may alternatively be another candidate ultrasound image determined according to ranking. In addition, the target ultrasound image may alternatively be a target ultrasound image determined by combining the region-of-interest detection result of the candidate ultrasound images with ranking of the candidate ultrasound images.

In step 1002, after the target ultrasound image is determined, the position of the ultrasonic transducer when the target ultrasound image is acquired may be used as the first position, and then the ultrasonic transducer is moved to the first position and maintained at the first position, thereby acquiring the second ultrasound image satisfying requirements in real time.

For example, when the ultrasonic transducer is at the first position, the area of a cross section of the region of interest in a scanning plane of the ultrasonic transducer is greater than a preset area threshold. For example, when the ultrasonic transducer is at the first position, the area of a cross section of the region of interest in a scanning plane corresponding to the first position is the greatest;

For another example, when the ultrasonic transducer is at the first position, the distance between two points on the boundary of a cross section of the region of interest in a scanning plane of the ultrasonic transducer is greater than a preset distance threshold. For example, when the ultrasonic transducer is at the first position, the distance between two points on the boundary of a cross section of the region of interest in a scanning plane corresponding to the first position is the greatest.

Hence, when the ultrasonic transducer is at the first position, it can be ensured that the scanning plane of the ultrasonic transducer is the optimal interventional guidance plane, which helps to guide the interventional object to the central position on the region of interest, thereby ensuring the effectiveness of the interventional procedure. In addition, the operator can easily observe the region of interest through the second ultrasound image, and a fault tolerance rate of the interventional procedure can be appropriately increased, reducing requirements for the interventional procedure. For example, the greatest cross section of the region of interest or the greatest distance between two points in the region of interest indicates that the range of a target position for the interventional procedure is the greatest, and even if there is a slight deviation during the intervention procedure, the interventional object is unlikely to fall outside the region of interest.

In step 404, real-time image recognition can be performed on the second ultrasound image to determine that the interventional object appears in a plane in which the second ultrasound image is located.

FIG. 12 is a schematic diagram of a second ultrasound image according to an embodiment of the present application. As shown in FIG. 12, a metal needle is used as an example of the interventional object; when a strong-echo point-like object A is identified in the second ultrasound image, it is determined that the interventional object appears in the plane in which the second ultrasound image is located. But the present application is not limited thereto, and it is also possible to use other methods to determine whether or not the interventional object appears in the plane in which the second ultrasound image is located.

In step 404, when the interventional object appears in the second ultrasound image, the interventional object may be stopped from moving (for example, a fourth drive member to be described later is stopped from driving the interventional object to move), and the position of the interventional object in the second ultrasound image when the interventional object appears in the second ultrasound image is used as the end position of the interventional object. Corresponding indication information is generated according to a positional relationship between the end position and the region of interest in the second ultrasound image.

For example, when the end position is within the region of interest, first indication information for indicating successful operation of the interventional object is generated; and for another example, when the end position is outside the region of interest, second indication information for indicating failed operation of the interventional object is generated. Thus, different indication information is generated for different situations, which helps the operator to accurately understand the result of the current interventional procedure.

The indication information may be information in various forms. For example, the indication information may be audio information and/or display information, or the like. The first indication information and the second indication information may be different audio information and/or display information.

In some embodiments, the controller may further determine a movement path of the interventional object according to the position information of the region of interest. The movement path includes a start position and a target position, the start position may be located outside a scanning plane corresponding to the first position, and the target position is located within the scanning plane corresponding to the first position.

Hence, the movement path of the interventional object can be automatically set according to the position information of the region of interest, which can reduce the dependence of the interventional procedure on the operator's experience, and enables the operator to quickly and accurately perform the interventional procedure. In addition, by configuring the start position of the movement path to be outside the scanning plane of the ultrasonic transducer, path planning can be performed more flexibly, which helps to reduce the risk of the interventional procedure and improve the success rate of the interventional procedure. The target position of the movement path is within the scanning plane of the ultrasonic transducer, thereby allowing the result of the interventional procedure to be reliably determined according to the second ultrasound image.

For example, the position information of the region of interest may include position information of the region of interest in the second ultrasound image. The target position of the movement path of the interventional object may be located within the region of interest of the second ultrasound image. For example, the target position may be at the center of the region of interest of the second ultrasound image. The start position of the movement path of the interventional object may be determined according to the target position, an intervention angle of the interventional object, and the like.

In some embodiments, the controller may further generate guidance information for indicating a positional relationship between a real-time position of the interventional object and the region of interest according to the first ultrasound images, an intervention angle of the interventional object, a start position of the interventional object, and an intervention distance of the interventional object within the object being scanned. Thus, the real-time position of the interventional object within the object being scanned can be accurately determined. By providing real-time guidance for the interventional procedure, the difficulty of the interventional procedure can be reduced and the accuracy and success rate of the interventional procedure can be improved.

The intervention angle of the interventional object, the start position of the interventional object, and the intervention distance of the interventional object within the object being scanned may be acquired in various manners. For example, if the interventional object is driven by a drive member to be described later, the intervention angle, the start position, and the intervention distance of the interventional object may be determined according to drive information or drive data of the drive member. But the present application is not limited thereto, and it is also possible to acquire images of the interventional object in real time by using a camera apparatus, and the interventional angle, the start position, the intervention distance, and the like are determined according to the images of the interventional object.

The guidance information may be generated in various manners. For example, when the guidance information is generated, image reconstruction may be performed based on the first ultrasound images to acquire a reconstructed image. A first coordinate system of the reconstructed image is registered with a second coordinate system in which the interventional object is located, so as to generate a transformation matrix representing a conversion relationship between the first coordinate system and the second coordinate system. According to the transformation matrix, the start position, interventional angle, intervention distance, and the like of the interventional object in the second coordinate system are transformed into the first coordinate system, and the travel path and the position of the interventional object in the first coordinate system are simulated and updated in real time. The guidance information is generated according to the position of the region of interest in the first coordinate system, and the real-time travel path and the real-time position of the interventional object in the first coordinate system.

The guidance information can be displayed on a display screen in a visual form, for example, guidance information based on a plurality of viewing angles can be displayed. In addition, the guidance information may also be provided to the operator through voice broadcast or the like. For example, when the interventional object is about to reach the depth of the cross section where the region of interest lies, a voice prompt may be issued: “the interventional object has reached the vicinity of the target cross section, the current depth is P millimeters away from the target depth, and the depth can be adjusted appropriately”.

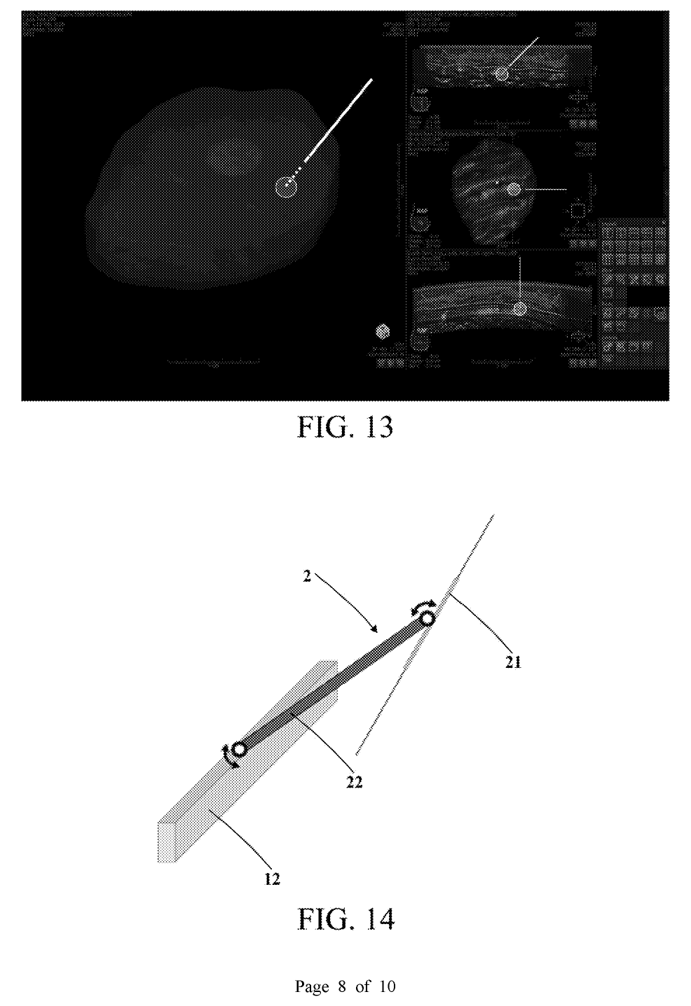

FIG. 13 is a schematic diagram of guidance information according to an embodiment of the present application. By using a breast as an example of the object being scanned and a puncture needle as an example of the interventional object, FIG. 13 shows guidance information from multiple viewing angels, including reconstructed images of the breast and a puncture needle. As shown on the left side of FIG. 13, by using three dimensional reconstruction technology, the operator can observe the positional relationship between the breast tissue and the puncture needle from a three-dimensional perspective. The guidance information in this viewing angle includes the three dimensional morphology and volume of the lesion (region of interest), and the trajectory of the puncture needle in the three-dimensional space. The operator can observe the positions of the puncture needle and the lesion from different angles by rotating and zooming the three-dimensional image, to ensure that the puncture path avoids structures such as major blood vessels and nerves.

As shown on the right side of FIG. 13, by using three dimensional reconstruction technology, the operator can observe the positional relationship between the breast tissue and the puncture needle from a coronal plane viewing angle, a sagittal plane viewing angle, and a transverse plane viewing angle. For example, according to the real-time position of the needle tip of the puncture needle, a coronal plane view, a sagittal plane view, and a transverse plane view corresponding to said real-time position can be displayed. Through the above information, the physician can accurately understand or adjust the direction and depth of the puncture needle in all viewing angles, so that the puncture needle can accurately reach the target position.

The position of the region of interest can be marked in the guidance information, thereby facilitating observation by the operator. In addition, in the guidance information, the real-time position of the interventional object and the position to be reached may further be represented through different images. For example, as shown in FIG. 13, the real-time position of the puncture needle is represented by a solid line, and the position that the puncture needle is about to reach (that is, the movement path predicted based on the current position and angle of the puncture needle) is represented by a dotted line.

In some embodiments, the housing of the ultrasound imaging assembly is fixed on the object being scanned during the scanning and intervention processes. Compared with a conventional ultrasound imaging assembly provided with a handheld probe, the present application allows the housing of the ultrasound imaging assembly to remain relatively fixed in position to the object being scanned during the scanning and intervention processes, thereby reducing operation difficulty and complexity, and improving the accuracy and success rate of the interventional procedure.

It should be noted that the above figures merely schematically illustrate the embodiments of the present application, but the present application is not limited thereto. For example, the order of execution between operations may be appropriately adjusted. In addition, some other operations may be added or some operations may be omitted. Those skilled in the art can make appropriate variations according to the above content, rather than being limited by the disclosure of the foregoing accompanying drawings.

The foregoing description relates only to the components or modules related to the present application, but the present application is not limited thereto. The ultrasound imaging system may further include other components or modules, or some components or modules may be omitted, and reference may be made to the related art for details of these components or modules.

In addition, for simplicity, the above figures only exemplarily illustrate connection relationships or signal directions between various components or modules, but it should be clear to those skilled in the art that various related technologies such as bus connection may be used. The various components or modules may be implemented by means of hardware facilities such as a processor, a memory, a transmitter and a receiver. The implementation of the present application is not limited thereto.

According to the above embodiment, an image sequence of an object being scanned is acquired by moving the ultrasonic transducer within the housing; position information of the region of interest is determined according to the image sequence; the ultrasonic transducer is automatically moved to a first position corresponding to the region of interest according to the position information and a second ultrasound image is acquired in real time at the first position; and indication information for indicating a positional relationship between an end position of the interventional object and the region of interest is generated according to the second ultrasound image. Hence, the ultrasonic transducer can be accurately moved to the first position corresponding to the region of interest, and when the interventional object reaches a scanning plane corresponding to the first position, the positional relationship between the end position of the interventional object and the region of interest can be reliably determined according to the second ultrasound image, thereby helping the operator to accurately understand the result of the interventional procedure.

An embodiment of the present application further provides an imaging method for an ultrasound imaging system. The ultrasound imaging system includes an ultrasound imaging assembly, and the ultrasound imaging assembly includes: a housing and an ultrasonic transducer movable within the housing. The imaging method includes: moving the ultrasonic transducer within the housing and acquiring an image sequence of an object being scanned, the image sequence including a plurality of parallel first ultrasound images; determining position information of a region of interest according to the image sequence; moving, according to the position information, the ultrasonic transducer to a first position corresponding to the region of interest and acquiring a second ultrasound image in real time; and generating, according to the second ultrasound image, indication information for indicating a positional relationship between an end position of an interventional assembly and the region of interest.

The ultrasound imaging system described in the embodiment of the present application can correspond to the ultrasound imaging system 1 described in the foregoing embodiments, and the imaging method described in the embodiment of the present application can correspond to the method described in the foregoing embodiments. For the specific content, reference may be made to the description in the foregoing embodiments.

An embodiment of the present application further provides a medical system. The medical system includes the ultrasound imaging system described in the foregoing embodiments, and the content thereof is incorporated here and will not be further described. The medical system further includes an interventional assembly including an interventional object.

In some embodiments, the interventional assembly may be connected to the ultrasound imaging assembly of the ultrasound imaging system. By connecting the interventional assembly to the ultrasound imaging assembly, the conversion relationship between an image coordinate system of the ultrasound imaging system and an interventional coordinate system of the interventional assembly can be simplified, which helps to reliably acquire the conversion relationship in a simple manner, thereby ensuring the accuracy and success rate of the interventional procedure.

When the interventional assembly is connected to the ultrasound imaging assembly, the interventional assembly may be connected to the ultrasonic transducer of the ultrasound imaging assembly. In this way, the conversion relationship between the image coordinate system and the interventional coordinate system can be further simplified, thereby reducing computational difficulty, improving computational efficiency, and further improving the accuracy and success rate of the interventional procedure.

Below, the interventional assembly in the present application is exemplarily described by taking an interventional assembly connected to the ultrasonic transducer as an example. It may be understood that the connection manner between the interventional assembly and the ultrasonic transducer is not limited thereto, and the interventional assembly and the ultrasonic transducer may alternatively be connected in other manners.

FIG. 14 is a schematic diagram of the structures of an ultrasonic transducer and an interventional assembly according to an embodiment of the present application. As shown in FIG. 14, an interventional assembly 2 includes an interventional object 21 and at least one free arm 22. One end of the free arm 22 is connected to the ultrasonic transducer 12 in the following manner: one end of the free arm 22 is movable in the length direction of the ultrasonic transducer 12 and is rotatable about a first axis parallel to the length direction. The other end of the free arm 22 is connected to the interventional object 21 in the following manner: the interventional object 21 is rotatable about a second axis parallel to the length direction.

The free arm 22 is connected to the ultrasonic transducer 12 and the interventional object 21 in the above manner. In this way, as long as the position of one end of the free arm 22 in the length direction of the ultrasonic transducer 12, the rotation angle of one end of the free arm 22 about the first axis, and the rotation angle of the interventional object 21 about the second axis are adjusted, the interventional object 21 can be moved from the start position to the target position along a pre-planned movement path, thereby simplifying the drive manner of the interventional assembly 2.

In some embodiments, the free arm 22 and the interventional object 21 may be disposed in the same plane, and the plane may be a plane perpendicular to the length direction of the ultrasonic transducer 12. Therefore, the conversion relationship between the interventional coordinate system and the image coordinate system can be further simplified, which helps to further simplify the drive manner of the interventional assembly 2, thereby helping to reliably move the interventional object 21 to the target position.

For example, by moving one end of the free arm 22 in the length direction of the ultrasonic transducer 12, the one end of the free arm 22 is moved to a position aligned with the region of interest of the second ultrasound image. More specifically, the one end of the free arm 22 is moved to a position aligned with the target position in the region of interest of the second ultrasound image.

Because the plane in which the free arm 22 and the interventional object 21 are located is perpendicular to the length direction of the ultrasonic transducer 12, and an image plane of the second ultrasound image is parallel to the length direction of the ultrasonic transducer 12, the plane in which the free arm 22 and the interventional object 21 are located is perpendicular to the image plane of the second ultrasound image. In this way, when one end of the free arm 22 is aligned with the region of interest of the second ultrasound image, the interventional object 21 is also aligned with the region of interest of the second ultrasound image.

In this case, by adjusting a first rotation angle of the free arm 22 about the first axis and a second rotation angle of the interventional object 21 about the second axis, the interventional object 21 can be moved from the start position to the target position along the pre-planned movement path.

In some embodiments, the first rotation angle and/or the second rotation angle may be related to the interventional angle of the interventional object, the size of the ultrasonic transducer, the size of the free arm, and the depth of the region of interest in the second ultrasound image.

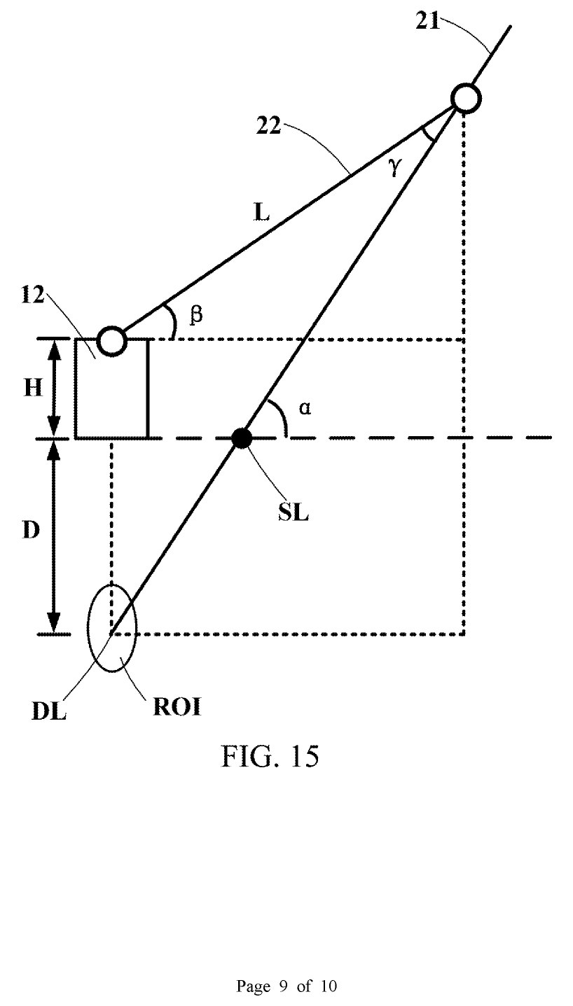

FIG. 15 is a schematic diagram of a positional relationship between an interventional assembly, an ultrasonic transducer, and a region of interest according to an embodiment of the present application. As shown in FIG. 15, the height of the ultrasonic transducer 12 is H, the depth of the target position DL within the region of interest ROI is D (that is, the distance between the target position DL and the surface of the object being scanned), the length of the free arm 22 is L, and the interventional angle of the interventional object 21 is α. When the above parameters are known, the first rotation angle β of the free arm 22 about the first axis and the second rotation angle γ of the interventional object 21 about the second axis can be determined through simple trigonometric calculations. Thus, the interventional object 21 is moved to the target position DL within the region of interest ROI from the start position SL on the surface of the object being scanned.

In some embodiments, the interventional assembly 2 may further include at least one of the following: a first drive member, a second drive member, a third drive member, and a fourth drive member. The first drive member can drive one end of the free arm 22 to move in the length direction of the ultrasonic transducer 12; the second drive member can drive one end of the free arm 22 to rotate about the first axis; the third drive member can drive the interventional object 21 to rotate about the second axis; and the fourth drive member can drive the interventional object 21 to move in the direction of extension of the interventional object 21.

Thus, the interventional object 21 can be automatically driven to move to the target position along the pre-planned movement path, thereby further simplifying the interventional procedure and ensuring the accuracy and reliability of the interventional procedure.

When the first drive member is provided, the first drive member can drive the free arm 22 to a position aligned with the region of interest. When the second drive member and/or the third drive member is provided, the drive angle of the second drive member and/or the third drive member may be related to at least one of the following parameters: an interventional angle of the interventional object, the size of the ultrasonic transducer, the size of the free arm, and the depth of the region of interest in the second ultrasound image. For the specific manner of determining the drive angle, reference may be made to the determination methods for the first rotation angle and the second rotation angle, which will not be described here.

The first drive member, the second drive member, the third drive member, and the fourth drive member may be in various forms. For example, the first drive member, the second drive member, the third drive member, and the fourth drive member may be stepper motors or the like.

In some embodiments, when the foregoing drive members are provided, the medical system may further be provided with a drive controller, which can be connected to the medical imaging system and the foregoing drive members to determine drive data of the respective drive members according to information output by the medical imaging system. The drive controller may also integrate the path planning function and/or guidance information generation function of the controller of the medical imaging system described above. In addition, the medical system may not require a separate drive controller, and the foregoing functions of the drive controller may be integrated into the controller of the medical imaging system described above.

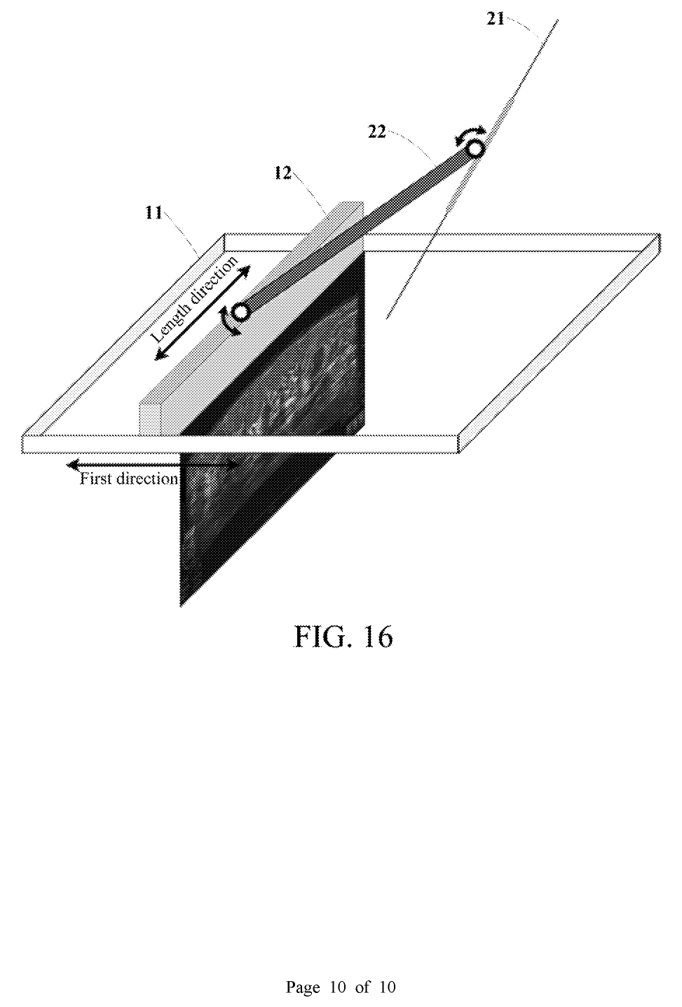

A control manner of the interventional assembly in the present application will be exemplarily described below with reference to the accompanying drawings by using a puncture needle as an example of the interventional object. FIG. 16 is a schematic diagram of an interventional assembly, a housing, an ultrasonic transducer, and a second ultrasound image according to an embodiment of the present application. As shown in FIG. 16, the ultrasonic transducer 12 can reciprocate within the housing 11 in the first direction shown in the figure. After the first position corresponding to the region of interest is determined, the ultrasonic transducer 12 is moved to the first position, and the ultrasonic transducer 12 acquires the second ultrasound image in real time at the first position. According to the position of the region of interest in the second ultrasound image, the drive controller of the interventional assembly controls the first drive member to move one end (the lower end shown in FIG. 16) of the free arm 22 to a position aligned with the target position in the region of interest. In addition, the drive controller determines drive angles of the second drive member and the third drive member according to the positional relationship shown in FIG. 15, thereby maintaining the puncture needle 21 at a correct puncture angle and enabling the needle tip of the puncture needle to be located at the start position of the movement path. Then, the drive controller controls the fourth drive member to move the puncture needle 21 along the movement path, and when the needle tip of the puncture needle 21 is detected in the second ultrasound image, the fourth drive member stops moving the puncture needle.

The control manners of the first drive member, the second drive member, the third drive member, and the fourth drive member are exemplarily described above, but the present application is not limited thereto. The first drive member, the second drive member, the third drive member, and the fourth drive member may alternatively be controlled in other manners, or at least one of the foregoing drive members may be omitted.

In some embodiments, the interventional assembly 2 may include an optical indicator. The optical indicator is configured to mark a start position on the surface of the object being scanned. Thus, the drive manner of the interventional assembly can be further simplified, improving the accuracy of the interventional procedure.

The optical indicator may mark the start position in various manners. For example, the optical indicator may be disposed at the other end of the free arm 22 (that is, the end to which the interventional object is connected), and the start position is marked on the surface of the object being scanned by using a light beam directed toward the object being scanned. When the light beam is aligned with the other end of the free arm 22 and the start position, the accuracy of the interventional angle of the interventional object 21 can be ensured by making the direction of extension of the interventional object 21 coincide with the light beam. Because the light beam provides a clear visual reference for the operator, the interventional object 21 can be easily guided to the correct interventional angle and start position.

When the optical indicator is provided, the third drive member for driving the interventional object 21 to rotate about the second axis may be omitted. The present application is not limited thereto. The third drive member may alternatively be retained in the interventional assembly 2, and whether the drive angle of the third drive member is accurate can be easily and reliably determined through the interventional angle and the start position marked by the optical indicator.

The optical indicator may be in various forms, such as a laser indicator.

The above description is made by taking an interventional assembly connected to the ultrasonic transducer as an example. But the present application is not limited thereto. When the interventional assembly is connected to the ultrasound imaging assembly, the interventional assembly may also be connected to other components of the ultrasound imaging assembly, for example, connected to the housing of the ultrasound imaging assembly. In this case, the conversion relationship between the image coordinate system of the ultrasound imaging system and the interventional coordinate system of the interventional assembly can be determined according to the position of the ultrasonic transducer in the housing.

In some embodiments, when the interventional assembly is connected to the housing of the ultrasound imaging assembly, the interventional assembly may be connected to a side wall of the housing, and the interventional assembly is movable along the side wall. The side wall may be parallel to the length direction of the ultrasonic transducer (that is, parallel to the image plane of the first ultrasound image or the second ultrasound image). Hence, the interventional assembly can be moved to a position aligned with the region of interest in the second ultrasound image, so that drive data of the interventional assembly can be determined in a simple manner, reducing computational difficulty and ensuring computational accuracy. But the present application is not limited thereto, and the interventional assembly may alternatively be connected to the housing of the ultrasound imaging assembly in other manners.

In some embodiments, the interventional assembly may also be disposed independently of the ultrasound imaging assembly of the ultrasound imaging system, that is, the interventional assembly is not connected to the ultrasound imaging assembly of the ultrasound imaging system. For example, the interventional assembly may be connected to other components of the ultrasound imaging system, or the interventional assembly may not be connected to the ultrasound imaging system.

According to the above embodiment, in the medical system, an image sequence of an object being scanned is acquired by moving the ultrasonic transducer within the housing; position information of the region of interest is determined according to the image sequence; the ultrasonic transducer is automatically moved to a first position corresponding to the region of interest according to the position information and a second ultrasound image is acquired in real time at the first position; and indication information for indicating a positional relationship between an end position of the interventional object and the region of interest is generated according to the second ultrasound image. Hence, the ultrasonic transducer can be accurately moved to the first position corresponding to the region of interest, and when the interventional object reaches a scanning plane corresponding to the first position, the positional relationship between the end position of the interventional object and the region of interest can be reliably determined according to the second ultrasound image, thereby helping the operator to accurately understand the result of the interventional procedure.

An embodiment of the present application further provides a computer-readable program, wherein the program, when executed, causes a computer to perform, in an ultrasound imaging system or a medical system, the method described in the foregoing embodiments.

An embodiment of the present application further provides a storage medium having a computer-readable program stored therein, wherein the computer-readable program causes a computer to perform, in an ultrasound imaging system or a medical system, the method described in the foregoing embodiments.

An embodiment of the present application further provides a computer program product at least including a computer program, wherein the computer program, when executed by a processor, causes an ultrasound imaging system or a medical system to perform the control method described in the foregoing embodiments.

The foregoing system, apparatus, and method of the present application can be implemented by hardware or by hardware in combination with software. The present application relates to such a computer-readable program that when executed by a logic component, the program causes the logic component to implement the foregoing apparatus or a constituent component, or causes the logic component to implement various methods or steps as described above. The present application further relates to a storage medium for storing the above program, such as a hard disk, a disk, an optical disk, a DVD, a flash memory, etc.

The method/apparatus described in view of the embodiments of the present application may be directly embodied as hardware, a software module executed by a processor, or a combination of the two. For example, one or more of the functional block diagrams and/or one or more combinations of the functional block diagrams shown in the drawings may correspond to either respective software modules or respective hardware modules of a computer program flow. The foregoing software modules may respectively correspond to the steps shown in the figures. The foregoing hardware modules can be implemented, for example, by firming the software modules using a field-programmable gate array (FPGA).

The software modules may be located in a RAM, a flash memory, a ROM, an EPROM, an EEPROM, a register, a hard disk, a portable storage disk, a CD-ROM, or any other form of storage medium known in the art. The storage medium may be coupled to a processor, so that the processor can read information from the storage medium and can write information into the storage medium. Alternatively, the storage medium may be a constituent component of the processor. The processor and the storage medium may be located in an ASIC. The software modules may be stored in a memory, and may alternatively be stored in a removable memory card. For example, if the device uses a large-capacity MEGA-SIM card or a large-capacity flash memory apparatus, the software modules can be stored in the MEGA-SIM card or the large-capacity flash memory apparatus.

One or more of the functional blocks and/or one or more combinations of the functional blocks shown in the accompanying drawings may be implemented as a general-purpose processor, a digital signal processor (DSP), an application-specific integrated circuit (ASIC), a field-programmable gate array (FPGA) or other programmable logic devices, discrete gate or transistor logic devices, a discrete hardware assembly, or any appropriate combination thereof for executing the functions described in the present application. The one or more functional blocks and/or the one or more combinations of the functional blocks shown in the accompanying drawings may also be implemented as a combination of computing devices, such as a combination of a DSP and a microprocessor, multiple microprocessors, one or more microprocessors in communication combination with a DSP, or any other such configuration.

The present application is described above with reference to specific implementations. However, it should be clear to those skilled in the art that the foregoing description is merely illustrative and is not intended to limit the scope of protection of the present application. Various variations and modifications may be made by those skilled in the art according to the spirit and principle of the present application, and these variations and modifications also fall within the scope of the present application. For example, each of the above embodiments may be used independently, or one or more of the above embodiments may be combined.

Preferred implementations of the present application are described above with reference to the accompanying drawings. Many features and advantages of the implementations are clear according to the detailed description. Therefore, the appended claims are intended to cover all these features and advantages that fall within the true spirit and scope of these implementations. In addition, as many modifications and changes could be easily conceived of by those skilled in the art, the implementations of the present application are not limited to the illustrated and described precise structures and operations, but can encompass all appropriate modifications, changes, and equivalents that fall within the scope of the implementations.

1. An ultrasound imaging system for guiding an interventional object, the system comprising an ultrasound imaging assembly and a controller, and the ultrasound imaging assembly comprising a housing and an ultrasonic transducer movable within the housing,
wherein the controller is configured to:
move the ultrasonic transducer within the housing and acquiring an image sequence of an object being scanned, the image sequence comprising a plurality of parallel first ultrasound images;
determine position information of a region of interest according to the image sequence;
move, according to the position information, the ultrasonic transducer to a first position corresponding to the region of interest and acquiring a second ultrasound image in real time; and
generate, according to the second ultrasound image, indication information for indicating a positional relationship between an end position of the interventional object and the region of interest.

2. The ultrasound imaging system according to claim 1, wherein the controller is further configured to:
determine a movement path of the interventional object according to the position information of the region of interest, wherein the movement path comprises a start position and a target position, the start position is located outside a scanning plane corresponding to the first position, and the target position is located within the scanning plane corresponding to the first position.

3. The ultrasound imaging system according to claim 1, wherein the determining position information of a region of interest according to the image sequence comprises:
generating, according to the image sequence, a third ultrasound image having an image plane direction different from that of the first ultrasound images; and
determining the position information of the region of interest according to the third ultrasound image.

4. The ultrasound imaging system according to claim 3, wherein
the first ultrasound images are perpendicular to a plane in which the object being scanned is located; or
the third ultrasound image is parallel to the plane in which the object being scanned is located.

5. The ultrasound imaging system according to claim 1, wherein the controller is further configured to:
according to the position information of the region of interest, determine the first position in the following manner:
according to the position information of the region of interest, select, from the image sequence, first ultrasound images comprising the region of interest as candidate ultrasound images; and
determine a target ultrasound image from the candidate ultrasound images, and using a position of the ultrasonic transducer corresponding to the target ultrasound image as the first position.

6. The ultrasound imaging system according to claim 5, wherein the determining a target ultrasound image from the candidate ultrasound images comprises:
performing detection on the candidate ultrasound images for the region of interest, and determining the target ultrasound image according to a detection result; or
determining the target ultrasound image from the candidate ultrasound images according to ranking of the candidate ultrasound images.

7. The ultrasound imaging system according to claim 1, wherein
the first position of the ultrasonic transducer comprises the following position:
based on the ultrasonic transducer being at said position, the area of a cross section of the region of interest in a scanning plane of the ultrasonic transducer is greater than a preset area threshold; or
based on the ultrasonic transducer being at said position, the distance between two points on the boundary of a cross section of the region of interest in a scanning plane of the ultrasonic transducer is greater than a preset distance threshold.

8. The ultrasound imaging system according to claim 1, wherein
based on the ultrasonic transducer being at the first position, the area of a cross section of the region of interest in a scanning plane corresponding to the first position is the greatest; or
based on the ultrasonic transducer being at the first position, the distance between two points on the boundary of a cross section of the region of interest in a scanning plane corresponding to the first position is the greatest.

9. The ultrasound imaging system according to claim 1, wherein the controller is further configured to:
perform real-time image recognition on the second ultrasound image, and determining that the interventional object appears in a plane in which the second ultrasound image is located.

10. The system according to claim 9, wherein the controller is further configured to:
based on the end position being within the region of interest, generating first indication information for indicating successful operation of the interventional object; or
based on the end position being outside the region of interest, generating second indication information indicating failed operation of the interventional object.

11. A medical system, comprising:
an ultrasound imaging system for guiding an interventional object, the system comprising an ultrasound imaging assembly and a controller, and the ultrasound imaging assembly comprising a housing and an ultrasonic transducer movable within the housing; and
an interventional assembly comprising the interventional object
wherein the controller is configured to:
move the ultrasonic transducer within the housing and acquiring an image sequence of an object being scanned, the image sequence comprising a plurality of parallel first ultrasound images;
determine position information of a region of interest according to the image sequence;
move, according to the position information, the ultrasonic transducer to a first position corresponding to the region of interest and acquiring a second ultrasound image in real time; and
generate, according to the second ultrasound image, indication information for indicating a positional relationship between an end position of the interventional object and the region of interest.

12. The system according to claim 11, wherein
based on the interventional object appearing in the second ultrasound image, the interventional object stops moving, and the position of the interventional object in the second ultrasound image when the interventional object appears in the second ultrasound image is used as the end position of the interventional object.

13. The system according to claim 11, wherein
the interventional assembly is connected to the ultrasound imaging assembly; or
the interventional assembly and the ultrasound imaging assembly are disposed independently of each other.

14. The system according to claim 13, wherein
the interventional assembly is connected to the ultrasonic transducer or the housing of the ultrasound imaging assembly.

15. The system according to claim 14, wherein
the interventional assembly further comprises at least one free arm,
wherein one end of the free arm is connected to the ultrasonic transducer in the following manner: one end of the free arm is movable in a length direction of the ultrasonic transducer and is rotatable about a first axis parallel to the length direction, and
the other end of the free arm is connected to the interventional object in the following manner: the interventional object is rotatable about a second axis parallel to the length direction.

16. The system according to claim 15, wherein
the interventional assembly further comprises at least one of the following:
a first drive member configured to drive one end of the free arm to move in the length direction of the ultrasonic transducer;
a second drive member configured to drive one end of the free arm to rotate about the first axis;
a third drive member configured to drive the interventional object to rotate about the second axis;
a fourth drive member configured to drive the interventional object to move in a direction of extension of the interventional object; and
an optical indicator configured to mark a start position on a surface of the object being scanned.

17. The system according to claim 16, wherein
the first drive member drives the free arm to a position aligned with the region of interest.

18. The system according to claim 16, wherein
a drive angle of the second drive member and/or the third drive member is related to at least one of the following parameters:
an intervention angle of the interventional object, the size of the ultrasonic transducer, the size of the free arm, and the depth of the region of interest in the second ultrasound image.

19. The system according to claim 11, wherein the controller is further configured to perform the following:
according to the first ultrasound images, an intervention angle of the interventional assembly, a start position of the interventional assembly, and an intervention distance of the interventional assembly within the object being scanned, generating guidance information for indicating a positional relationship between a real-time position of the interventional assembly and the region of interest.

20. An imaging method for an ultrasound imaging system, the ultrasound imaging system comprising an ultrasound imaging assembly, and the ultrasound imaging assembly comprising a housing and an ultrasonic transducer movable within the housing; the method comprises:
moving the ultrasonic transducer within the housing and acquiring an image sequence of an object being scanned, the image sequence comprising a plurality of parallel first ultrasound images;
determining position information of a region of interest according to the image sequence;
moving, according to the position information, the ultrasonic transducer to a first position corresponding to the region of interest and acquiring a second ultrasound image in real time; and
generating, according to the second ultrasound image, indication information for indicating a positional relationship between an end position of an interventional assembly and the region of interest.
